(19) United States
(12) Patent Application Publication
(10) Pub. No.: US 2026/0132461 A1
(43) Pub. Date: May 14, 2026

(54) MOLECULAR MARKER ASSOCIATED WITH IMMUNE PARAMETERS IN YAKS AND USE THEREOF IN BREEDING

(71) Applicant: SERES ACADEMIAE AGRICULTURAE SCIENTIARUM LANZHOU INSTITUTUM ANIMAL PHARMACEUTICAL ET VETERINARII, Lanzhou (CN)

(72) Inventors: Chunnian LIANG, Lanzhou (CN); Daoning YU, Lanzhou (CN); Xue ZHAO, Lanzhou (CN); Yan YANG, Lanzhou (CN); Xiaoming MA, Lanzhou (CN); Xita ZHA, Lanzhou (CN); Yongfu LA, Lanzhou (CN); Xiaoyun WU, Lanzhou (CN); Min CHU, Lanzhou (CN); Yuansheng ZHAO, Lanzhou (CN); Xuemei LIU, Lanzhou (CN)

(73) Assignee: SERES ACADEMIAE AGRICULTURAE SCIENTIARUM LANZHOU INSTITUTUM ANIMAL PHARMACEUTICAL ET VETERINARII, Lanzhou (CN)

(21) Appl. No.: 19/414,317

(22) Filed: Dec. 10, 2025

Related U.S. Application Data
(63) Continuation of application No. PCT/CN2025/114576, filed on Aug. 14, 2025.

(30) Foreign Application Priority Data
Nov. 8, 2024 (CN) 202411588353.8

(51) Int. Cl. C12Q 1/6876

(52) U.S. Cl. CPC C12Q 1/6876; C12Q 2600/124; C12Q 2600/156

(57) ABSTRACT

A molecular marker associated with immune parameters in yaks and use thereof in breeding are provided. A single nucleotide polymorphism (SNP) molecular marker associated with immune traits in yaks is identified through screening. The SNP molecular marker is located at position 26593056 on chromosome 3 of the yak reference genome version LU_Bosgru_v3.0, and a nucleotide at the position is G or A. The SNP molecular marker is utilized to quantify levels of immunoglobulin A, immunoglobulin G, and immunoglobulin M in a yak individual, providing a novel SNP molecular marker resource for non-diagnostic, marker-assisted selection of immune traits in yaks, thereby providing a basis for breeding yaks with enhanced immune competence.

### CROSS REFERENCE TO THE RELATED APPLICATIONS

This application is a continuation application of International Application No. PCT/CN2025/114576, filed on Aug. 14, 2025, which is based upon and claims priority to Chinese Patent Application No. 202411588353.8, filed on Nov. 8, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBRZBC335_Sequence_Listing.xml, created on Nov. 25, 2025, and is 4,038 bytes in size.

### TECHNICAL FIELD

The present invention relates to the field of molecular biology detection technology, and more particularly, to a molecular marker associated with immune parameters in yaks and use thereof in breeding.

### BACKGROUND

Yaks, inhabiting high-altitude regions over extended periods, are exposed to adverse environmental conditions and must possess high immunity and disease resistance to adapt to stressors including, but not limited to, high altitude, low atmospheric pressure, intense ultraviolet radiation, hypoxia, and nutrient deficiency. In the yak farming industry, disease conditions pose severe threats to animal health, resulting in substantial economic losses. Despite the implementation of measures such as improved feeding management, pharmaceutical interventions, and vaccination programs to prevent diseases to some extent, these methods have proven ineffective in controlling the transmission of infectious diseases. Moreover, the extensive use of pharmaceuticals risks the development of drug resistance and poses potential safety concerns for animal-derived products. Therefore, from a long-term perspective, conducting disease-resistant breeding through genetic screening to enhance the disease resistance of yaks is the fundamental approach to resolving the issue.

Immune parameters serve as critical indicators reflecting the disease resistance capability of yaks, depending on the health status and function of the immune system. The primary function of the immune system is to identify and eliminate invading pathogens. Upon invasion by pathogens, the immune system responds rapidly by initiating a series of immune processes to combat and eliminate the pathogens. Immune parameters such as antibody levels and immunoglobulin levels serve as key indicators for assessing animal health and disease resistance capabilities. By monitoring changes in immune parameters, immune issues in yaks can be timely identified, enabling the implementation of measures to enhance disease resistance, thereby preventing and treating diseases.

Immunoglobulins are a group of proteins possessing antibody activity, predominantly present in blood plasma, and additionally found in other bodily fluids, tissues, and certain secretions. Immunoglobulins are classified into five classes, namely Immunoglobulin G (IgG), Immunoglobulin A (IgA), Immunoglobulin M (IgM), Immunoglobulin D (IgD), and Immunoglobulin E (IgE). The immunoglobulins are critically associated with disease resistance in animals. For instance, a decrease in the levels of IgG and IgA in vivo is associated with an increased susceptibility to immunodeficiency. An increase in the levels of IgG and IgA in vivo is correlated with enhanced disease resistance in the organism. Enhanced disease resistance thereby reduces medication usage, decreases production costs, and ultimately increases farming/aquaculture benefits. Therefore, immunoglobulins can serve as an immune parameter. Changes in immunoglobulins enable the timely detection of immune issues in yaks, and allow for the adoption of corresponding measures to enhance their disease resistance, thereby preventing and treating diseases.

With the rapid development of molecular marker technology, utilizing molecular markers for early-stage selection in yak breeding to screen for individuals exhibiting high disease resistance has become an important approach to enhance breeding efficiency and accuracy. Through screening for genetic variation sites and association analysis with disease-resistant traits, relationships between relevant genes and immune traits are identified, thereby promoting the advancement of disease-resistant breeding. The discovery provides novel approaches and genetic resources for breeding yaks with enhanced disease resistance, offering significant practical value.

### SUMMARY

An objective of the present invention is to provide a single nucleotide polymorphism (SNP) molecular marker associated with immune parameters in yaks and use thereof.

To achieve the above objective, the present invention provides the following technical solutions.

The present invention provides an SNP molecular marker associated with immune traits in yaks, wherein the SNP molecular marker is located at position 26593056 on chromosome 3 of the yak reference genome version LU_Bosgru_v3.0, and a nucleotide at the position is G or A.

Preferably, for a yak genotype where the nucleotide is G, the genotype is GG or GA; and for a yak genotype where the nucleotide is A, the genotype is AA; a yak individual with the genotype GG exhibits a higher level of immunoglobulin A (IgA) than a yak individual with the genotype GA or the genotype AA; and a yak individual with the genotype GG and GA exhibits a higher level of immunoglobulin G (IgG) and immunoglobulin M (IgM) than a yak individual with the genotype AA.

The present invention further provides use of the SNP molecular marker in preparing a product for assessing immune competence in yaks, or in preparing a product for marker-assisted breeding in yaks.

The present invention further provides a primer pair for amplifying the SNP molecular marker, wherein the sequences of the primer pair are set forth as SEQ ID NO: 1 and SEQ ID NO: 2.

The present invention further provides use of the primer pair in preparing a product for detecting immunity in yaks, or in preparing a product for marker-assisted breeding in yaks.

The present invention further provides a kit for assessing immune competence in yaks, including a reagent for detecting the SNP molecular marker or the primer pair.

The present invention further provides a kit for marker-assisted breeding in yaks, including a reagent for detecting the SNP molecular marker or the primer pair.

The present invention further provides a method for non-diagnostic, marker-assisted selection of immune traits in yaks, including the steps of:
(1) extracting yak genomic DNA;
(2) using the yak genomic DNA obtained in step (1) as a template, amplifying with the primer pair to obtain an amplification product; and
(3) performing a genotypic analysis on the amplification product to characterize yaks with different genotypes; and correlating genotypes of yaks with immune parameters, wherein an immunoglobulin is selected from the group consisting of IgA, IgG, IgM, and a combination thereof.

Preferably, in step (2), an amplification system has a total volume of 25 μL, including: 12.5 μL of 2×L-Exp Taq Master Mix, 8.5 μL of RNase-free water, 1 μL of a forward primer, 1 μL of a reverse primer, and 2 μL of a template.

Preferably, in step (2), an amplification procedure includes: 94° C. for 1 min; 98° C. for 10 s, 58° C. for 30 s, 72° C. for 1 min, for 35 cycles; and a final extension at 72° C. for 2 min.

Compared with the prior art, the present invention has the following advantageous effects:

The present invention provides an SNP molecular marker associated with immune parameters in yaks and use thereof in breeding. The present invention has identified through research that the SNP locus associated with immunity in yaks is located at position 26593056 on chromosome 3 of the yak reference genome version LU_Bosgru_v3.0, with a nucleotide of G/A, and three genotypes are present: when the base at position 26593056 on chromosome 3 is G, the genotype is GG or GA; and when the base at position 26593056 on chromosome 3 is A, the genotype is AA.

Through association analysis between different genotypes and the levels of IgA, IgG, and IgM, it is found that the level of IgA in a yak individual with the GG genotype is higher than that in a yak individual with the GA or AA genotype (p<0.05); and the levels of IgG and IgM in a yak individual with the GG and GA genotypes is higher than that in a yak individual with the AA genotype (p<0.05).

The present invention enables the determination of the levels of IgA, IgG, and IgM in a yak individual by detecting the nucleotide base at position 26593056 on chromosome 3. The present invention provides a novel SNP molecular marker resource for non-diagnostic, marker-assisted selection of immune traits in yaks, thereby providing a basis for breeding yaks with enhanced immune competence.

### BRIEF DESCRIPTION OF THE DRAWINGS

To more clearly illustrate the technical solutions in the examples of the present invention or the prior art, the following provides a brief description of the drawings used in the description of the examples or the prior art. It is to be understood that the drawings described below are merely for the examples of the present invention. For those of ordinary skill in the art, other drawings may be derived from the drawings without creative efforts.

FIG. 1 shows PCR amplification products; wherein Lane M represents the DNA marker; and Lanes 1, 2, and 3 represent three experimental replicates.

FIG. 2 shows the chromatogram and corresponding nucleotide sequence obtained after sequencing the PCR products.

### DETAILED DESCRIPTION OF THE EMBODIMENTS

The present invention provides an SNP molecular marker associated with immune parameters in yaks, wherein the SNP molecular marker is located at position 26593056 on chromosome 3 of the yak reference genome version LU_Bosgru_v3.0, and a nucleotide at the position is G or A.

In the present invention, for a yak genotype where the nucleotide is G, the genotype is GG or GA; and for a yak genotype where the nucleotide is A, the genotype is AA; a yak individual with the genotype GG exhibits a higher level of immunoglobulin A (IgA) than a yak individual with the genotype GA or the genotype AA; and a yak individual with the genotype GG and GA exhibits a higher level of immunoglobulin G (IgG) and immunoglobulin M (IgM) than a yak individual with the genotype AA.

The present invention further provides use of the SNP molecular marker in preparing a product for assessing immune competence in yaks or screening yak breeds with enhanced immunity.

The present invention further provides a primer pair for amplifying a gene fragment including the SNP molecular marker, wherein the nucleotide sequences of the primer pair are set forth in SEQ ID NO: 1 and SEQ ID NO: 2.

SEQ ID NO: 1 is the forward primer, with the sequence:

5′-CACACAGGAAGCAAAGGCAC-3′;

SEQ ID NO: 2 is the reverse primer, with the sequence:

5′-TAGGACAGCAAAGCCAGCAA-3′

The present invention further provides use of the primer pair in preparing a product for detecting immunity in yaks, or in preparing a product for marker-assisted breeding in yaks.

The present invention further provides a kit for assessing immune competence in yaks, including a reagent for detecting the SNP molecular marker or the primer pair.

The present invention further provides a kit for marker-assisted breeding in yaks, including a reagent for detecting the SNP molecular marker or the primer pair.

The present invention further provides a method for non-diagnostic, marker-assisted selection of immune traits in yaks, including the steps of:
(1) yak genomic DNA is extracted;
(2) the yak genomic DNA obtained in step (1) is used as a template, amplification is performed with the primer pair, to obtain an amplification product; and
(3) a genotypic analysis is performed on the amplification product to characterize yaks with different genotypes; and correlating genotypes of yaks with immune parameters, wherein an immunoglobulin is selected from the group consisting of IgA, IgG, Ig M, and a combination thereof.

In the present invention, in step (2), an amplification system has a total volume of 25 μL, including: 12.5 μL of 2×L-Exp Taq Master Mix, 8.5 μL of RNase-free water, 1 μL of a forward primer, 1 μL of a reverse primer, and 2 μL of a template.

In the present invention, in step (2), an amplification procedure includes: 94° C. for 1 min; 98° C. for 10 s, 58° C. for 30 s, 72° C. for 1 min, for 35 cycles; and a final extension at 72° C. for 2 min.

The following describes the technical solutions provided by the present invention in conjunction with examples. However, the descriptions should not be construed as limiting the scope of protection of the present invention.

### Example 1

### 1. Sample Collection

The Nyangya yak breed was utilized as the detection subject in the present invention. Blood samples (5 mL each) were collected from 192 fasted yaks in pasture areas of Lhari County, Nagqu City, Tibet Autonomous Region, into clean clot activator vacuum blood collection tubes and allowed to stand for 30 min. Then the blood samples were centrifuged at 3500 r/min for 10 min. The resulting supernatant was aspirated into polyethylene (PE) tubes, which were subsequently sealed and stored at −20° C. in a low-temperature freezer. Additionally, 192 samples of blood (5 mL each) were collected into vacuum blood collection tubes containing EDTA-K2 anticoagulant. Following blood collection, each sample was immediately mixed thoroughly, placed into an insulated container containing ice packs for temporary storage, transported to the laboratory, and subsequently stored frozen at approximately −20° C. for subsequent extraction of genomic DNA.

### 2. Main Reagents and Apparatus

EDTA-K2 vacuum blood collection tubes were purchased from Jiangsu Yuli Medical Equipment Co., Ltd. A blood genomic DNA extraction kit was purchased from Tiangen Biotech (Beijing) Co., Ltd. A NanoDrop 2000 spectrophotometer was purchased from Thermo Fisher Scientific, USA. DL2000 DNA Marker, agarose, and nucleic acid dye were purchased from Solarbio Science & Technology Co., Ltd. (Beijing). 2×L-Exp Taq Master Mix (dye plus) was purchased from Hunan Accurate Bioengineering Co., Ltd. An electrophoresis apparatus was obtained from Beijing Liuyi Instrument Factory. A PCR instrument was obtained from Bio-Rad. IgA (MB-4907A), IgG (MB-4616A), and IgM (MB-4908A) detection kits were purchased from Jiangsu Meibiao Biotechnology Co., Ltd.

### 3. Methods

### 3.1 Detection of Immunoglobulins IgA, IgG, and IgM

Detection of IgA, IgG, and IgM was performed using a one-step sandwich immunoassay with two antibodies, implemented with commercially available assay kits from Jiangsu Meibiao Biotechnology Co., Ltd. First, the required test strips are removed from an aluminum foil bag equilibrated at room temperature for 20 min, and the remaining strips are sealed in a resealable bag and stored at 4° C. Standard wells and sample wells were arranged: into the standard wells, 50 μL of standard solutions at different concentrations were added respectively; into the sample wells, 10 μL of a test sample was first added, followed by the addition of 40 μL of a sample diluent; and no solution was added to blank wells. Except for the blank wells, 100 μL of horseradish peroxidase (HRP)-labelled detection antibody was added to each standard well and sample well.

The reaction wells were sealed with a plate sealer and incubated at 37° C. in a water bath or incubator for 60 min. The liquid was discarded and the wells were blotted dry on absorbent paper. Each well was then filled to capacity with wash buffer, allowed to stand for 1 min, followed by removal of the wash buffer and blotting dry on absorbent paper. The washing procedure was repeated five times (alternatively, the washing step was performed using an automated plate washer). Subsequently, 50 μL of Substrate A and 50 μL of Substrate B were added to each well, followed by incubation at 37° C. in the dark for 15 min. 50 μL of stop solution was added to each well. Within 15 min, the optical density (OD) value of each well was measured at a wavelength of 450 nm. Finally, a standard curve was plotted: in a spreadsheet software, a linear regression curve of the standard was generated with the standard concentration as the x-axis and the corresponding OD value as the y-axis. The concentrations of IgA, IgG, and IgM in each test sample were calculated according to the curve equation.

3.2 Extraction of Genomic DNA from Blood Samples

Genomic DNA was extracted from blood samples using the blood genomic DNA extraction kit of Tiangen Biotech (Beijing) Co., Ltd. The extracted DNA was analyzed by ultraviolet spectrophotometry to determine concentration and purity. DNA samples with concentrations exceeding 20 ng/μL and OD₂₆₀/OD₂₈₀ between 1.7 and 1.9 were considered acceptable for experimental use. These qualified samples were stored at approximately −20° C. for subsequent use.

### 3.3 Primer Design

Gene-specific primers encompassing the g.26593056G>A SNP locus were designed by accessing the Pick Primers online tool (available at the NCBI website), wherein the primer design was based on the reference sequence of Chromosome 3 from the yak genome assembly LU_Bosgru_v3.0 (GenBank accession no.: GCA_005887515.1).

The primer sequences were:

Forward primer:
(SEQ ID NO: 1)
5′-CACACAGGAAGCAAAGGCAC-3′;
and
Reverse primer:
(SEQ ID NO: 2)
5′-TAGGACAGCAAAGCCAGCAA-3′.

The amplified fragment length was 389 bp, and the primers were synthesized by Beijing Tsingke Biotechnology Co., Ltd.

### 3.4 PCR Amplification and Sequencing

PCR amplification system: a total volume of 25 μL, including: 12.5 μL of 2×L-Exp Taq Master Mix, 8.5 μL of RNase-free water, 1 μL of a forward primer, 1 μL of a reverse primer, and 2 μL of a template.

PCR amplification procedure: 94° C. for 1 min; 98° C. for 10 s, 58° C. for 30 s, 72° C. for 1 min, for 35 cycles; and a final extension at 72° C. for 2 min.

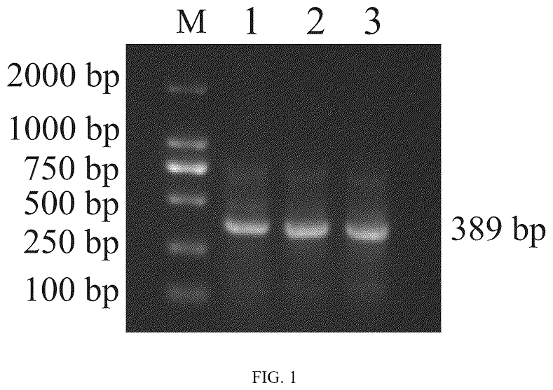

The PCR products were analyzed by 1% agarose gel electrophoresis. Following confirmation of the PCR products by agarose gel electrophoresis, the PCR products were subjected to direct sequencing, and the sequencing was performed by Beijing Tsingke Biotechnology Co., Ltd. Electrophoresis results were depicted in FIG. 1. As shown in FIG. 1, the PCR amplification produced a sequence with a length of 389 bp. Sequencing revealed a G/A substitution at position 26593056 on chromosome 3 of the Bos gru genome (version LU_Bosgru_v3.0) within an amplification product of 189 nucleotides. The amplification product exhibited clear bands with no non-specific bands, demonstrating high specificity. The locus was preliminarily identified as an SNP marker in yaks and designated as the g.26593056G>A SNP. The sequence obtained by CR amplification was shown as SEQ ID NO: 3, wherein a G substitution was present at position 189. The PCR amplification product exhibited a fragment size corresponding to the expected size, and was suitable for proceeding to the next experimental step.

SEQ ID NO: 3
CACACAGGAAGCAAAGGCACCTCAGGTTCCTAAATGAGGAATCAAG
TTAACATTCCCATTCTCCTCTATTGAGTTATCATTTTTGTCCCAT
TTTGTTCTCCTTGTCCCAGTGCCTTACCCACCACATACACACTGT
TACTCTGAGCTGAAACCCTTGCAGGGCATTAGCAGTCACACACCT
GACCACTGCCTTCTATCATCATACATTAATGCTCTTTTAACATTT
ATTTATTTTTAATTGGAAGATAATTGCTTTACAATGTTGTGGTTG
GTTTCTGCCATACATCAACATGAATCATTCATAGGTATACATATA
TCCCCTCTCTCTTGAACCTCCTTCATCATATACTTTTGTGATAAA
ATAGAAAATTGCTGGCTTTGCTGTCCTA

The sequencing results of the PCR products were aligned using bioanalysis software MEGA 11.0, the sequence chromatograms were analyzed, and genotyping was completed.

### 4. Statistical Analysis

Based on the genotyping results, the number of individuals for each genotype at each locus was counted. Gene frequency, genotype frequency, effective number of alleles (Ne), expected heterozygosity (He), and Hardy-Weinberg equilibrium testing for the genetic locus g26593056G>A were calculated using Popgen32 software. Polymorphism information content (PIC) was determined by dedicated PIC calculation software. The association between different yak genotypes and immunoglobulins IgA, IgG, and IgM was analyzed using the General Linear Model (GLM) in IBM SPSS Statistics 26 software. Results were presented as “mean±standard error”.

### 5. Results

### 5.1 Results of PCR Amplification and Sequencing

The amplification product of the g.26593056G>A SNP locus on chromosome 3 in yaks was detected using 1% agarose gel electrophoresis (as depicted in FIG. 1). The amplification product exhibited clear bands with no non-specific bands, demonstrating high specificity. The PCR amplification product exhibited a fragment size of 389 bp corresponding to the expected size, and was suitable for proceeding to the next experimental step.

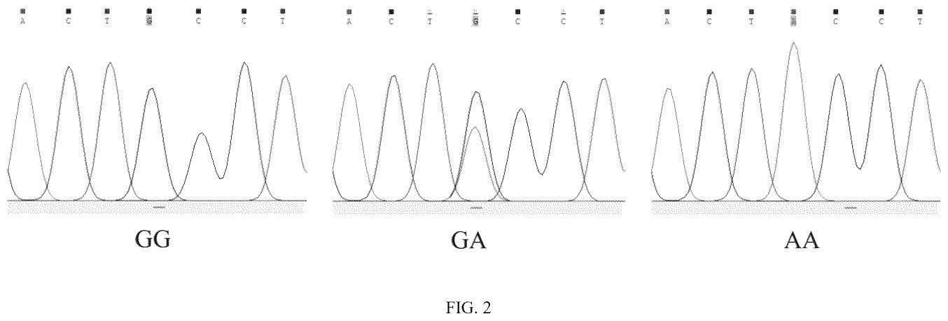

Following purification and sequencing of the PCR product, the resultant chromatogram and nucleotide sequence were presented in FIG. 2. Analysis of FIG. 2 demonstrated that a G-to-A substitution occurred at SNP locus g.26593056, wherein three genotypes (GG, GA, and AA) were detected.

### 5.2 Statistical Analysis Results

Genotype and allele frequencies at the g.26,593,056G>A SNP locus on chromosome 3 of Bos grunniens were analyzed from a population genetics perspective. As shown in Table 1, at the g.26593056G>A SNP locus, the GA genotype exhibited the highest frequency and was the predominant genotype. The G allele frequency was 54.2%, representing the predominant allele. Conformity with Hardy-Weinberg equilibrium was confirmed for the SNP locus by chi-square testing (P>0.05) (Table 1). An expected heterozygosity of 0.497 and polymorphism information content (PIC) value of 0.373 were calculated, wherein the PIC value within the range of 0.25 to 0.50 was classified as moderately polymorphic.

TABLE 1
Polymorphism Characteristics of the g.26593056G >
A SNP Locus on Chromosome 3 in Bos grunniens

| | | | | Allele | | | | | | |
|---|---|---|---|---|---|---|---|---|---|---|
| | Genotype Frequency | | | Frequency | | | | | | |
| Locus | GG | GA | AA | G | A | χ² | P | PIC | He | Ne |
| g26593056G > A | 0.307 | 0.469 | 0.224 | 0.542 | 0.458 | 0.601 | 0.438 | 0.373 | 0.497 | 1.986 |
| | (59) | (90) | (43) | | | | | | | |

### 5.3 Association Analysis Between Genotypes and Immunoglobulins IgA, IgG, and IgM

The association between different yak genotypes and the concentrations of immunoglobulins IgA, IgG, and IgM was analyzed using the General Linear Model procedure within IBM SPSS Statistics software, version 26. The results demonstrate that the level of IgA in a yak individual with the GG genotype was significantly higher than that in a yak individual with the GA or AA genotypes (p<0.05); and the levels of IgG and IgM in a yak individual with GG and GA genotypes were higher than that in a yak individual with the AA genotype (p<0.05). It was demonstrated that the base at position g.26593056G>A on chromosome 3 of the yaks was significantly associated (p<0.05) with IgA, IgG, and IgM in yaks. This base was thereby identified as the SNP marker for yaks. The results were shown in Table 2.

TABLE 2
Association Analysis Between Genotypes
and Immunoglobulins IgA, IgG, and IgM

| | Genotype | | |
|---|---|---|---|
| Trait | GG | GA | AA |
| IgA(μg/mL) | 1216.68 ± 31.21a | 1133.88 ± 14.93b | 1115.54 ± 30.80b |
| IgG(mg/mL) | 2.59 ± 0.09a | 2.40 ± 0.05a | 2.04 ± 0.11b |
| IgM(μg/mL) | 2188.09 ± 41.77a | 2124.34 ± 25.74a | 1983.65 ± 45.20b |

Note: within the same row, data points labelled with dissimilar lowercase letter superscripts indicate statistically significant differences (P<0.05)

The SNP molecular marker described in the present invention is located at position 26593056 on chromosome 3 of the yak reference genome version LU_Bosgru_v3.0, with a G/A nucleotide substitution, designated as g.26593056G>A, and three genotypes are present: when the base at position 26593056 on chromosome 3 is G, the genotype is GG or GA; and when the base at position 26593056 on chromosome 3 is A, the genotype is AA. Through association analysis between different genotypes and the levels of IgA, IgG, and IgM, it is found that the level of IgA in a yak individual with the GG genotype is higher than that in a yak individual with the GA or AA genotype (p<0.05); and the levels of IgG and IgM in a yak individual with the GG and GA genotypes is higher than that in a yak individual with the AA genotype (p<0.05). The present invention enables the determination of the levels of IgA, IgG, and IgM in a yak individual by detecting the nucleotide base at position 26593056 on chromosome 3. The present invention provides a novel SNP molecular marker resource for non-diagnostic, marker-assisted selection of immune traits in yaks.

The above descriptions are merely the preferred examples of the present invention. It is to be pointed out that those of ordinary skill in the art may also make several improvements and modifications without departing from the principle of the present invention, and such improvements and modifications shall fall within the scope of protection of the present invention.

What is claimed is:

1. A use of a reagent for detecting a single nucleotide polymorphism (SNP) molecular marker in preparing an in vitro detection reagent for immune traits in yaks, or preparing a detection reagent for immune traits marker-assisted breeding in yaks, wherein the SNP molecular marker is located at position 26593056 on chromosome 3 of a yak reference genome version LU_Bosgru_v3.0, and a nucleotide at the position is G or A;
for a yak genotype where the nucleotide is A, the genotype is AA; a yak individual with a genotype GG exhibits a higher level of immunoglobulin A (IgA) than a yak individual with a genotype GA or the genotype AA; and a yak individual with the genotype GG and GA exhibits a higher level of immunoglobulin G (IgG) and immunoglobulin M (IgM) than a yak individual with the genotype AA.

2. The use according to claim 1, wherein a primer pair for detecting the SNP molecular marker comprises the sequences set forth as SEQ ID NO: 1 and SEQ ID NO: 2.

3. A method for non-diagnostic, marker-assisted selection of immune traits in yaks, comprising steps of:
(1) extracting yak genomic DNA;
(2) using the yak genomic DNA obtained in step (1) as a template, amplifying with a primer pair to obtain an amplification product; and
(3) performing a genotypic analysis on the amplification product to characterize yaks with different genotypes; and correlating genotypes of yaks with immune parameters, wherein an immunoglobulin is selected from the group consisting of IgA, IgG, IgM, and a combination thereof;
the primer pair is used for amplifying an SNP molecular marker, and the SNP molecular marker is located at position 189 of SEQ ID NO: 3, and a nucleotide is G or A; wherein for a yak genotype where the nucleotide is A, the genotype is AA; a yak individual with a genotype GG exhibits a higher level of immunoglobulin A (IgA) than a yak individual with a genotype GA or the genotype AA; and a yak individual with the genotype GG and GA exhibits a higher level of immunoglobulin G (IgG) and immunoglobulin M (IgM) than a yak individual with the genotype AA.

4. The method according to claim 3, wherein in step (2), an amplification system has a total volume of 25 μL, comprising: 12.5 μL of 2× L-Exp Taq Master Mix, 8.5 μL of RNase-free water, 1 μL of a forward primer, 1 μL of a reverse primer, and 2 μL of the template.

5. The method according to claim 3, wherein in step (2), an amplification procedure comprises: 94° C. for 1 min; 98° C. for 10 s, 58° C. for 30 s, 72° C. for 1 min, for 35 cycles; and a final extension at 72° C. for 2 min.


SEQUENCE LISTING (3 sequences)

SEQ ID NO: 1 - DNA, 20 bp, synthetic construct
  source 1..20: mol_type = other DNA
cacacaggaagcaaaggcac

SEQ ID NO: 2 - DNA, 20 bp, synthetic construct
  source 1..20: mol_type = other DNA
taggacagcaaagccagcaa

SEQ ID NO: 3 - DNA, 389 bp, synthetic construct
  source 1..389: mol_type = other DNA
cacacaggaagcaaaggcacctcaggttcctaaatgaggaatcaagttaacattcccattctcctctattgagttatcatttttgtcccattttgttctccttgtcccagtgccttacccaccacatacacactgttactctgagctgaaacccttgcagggcattagcagtcacacacctgaccactgccttctatcatcatacattaatgctcttttaacatttatttatttttaattggaagataattgctttacaatgttgtggttggtttctgccatacatcaacatgaatcattcataggtatacatatatcccctctctcttgaacctccttcatcatatacttttgtgataaaatagaaaattgctggctttgctgtccta